(19) United States
(12) Patent Application Publication
(10) Pub. No.: US 2008/0004574 A1
(43) Pub. Date: Jan. 3, 2008

(54) SELECTABLE RATE INTRAVENOUS INFUSION SET

(76) Inventors: Daniel Dyar, Spencer, IN (US); Ladeana A. Dyar, Franklin, IN (US)

Correspondence Address: WOODARD, EMHARDT, MORIARTY, MCNETT & HENRY LLP, 111 MONUMENT CIRCLE, SUITE 3700, INDIANAPOLIS, IN, 46204-5137, US

(21) Appl. No.: 11/766,557

(22) Filed: Jun. 21, 2007

Related U.S. Application Data
(63) Continuation of application No. 11/551,944, filed on Oct. 23, 2006.
(60) Provisional application No. 60/729,139, filed on Oct. 21, 2005.

(51) Int. Cl. A61M 5/14

(52) U.S. Cl. 604/246

(57) ABSTRACT

An intravenous infusion device incorporates a valve which controls the intravenous fluid drip rate. First and second drip chambers are provided in which one drip chamber contains a macro drip nozzle and the other drip chamber contains a micro drip nozzle. The valve is selectable between a first position that connects an intravenous fluid bag to the macro drip nozzle and a second position that connects the fluid bag to the micro drip nozzle.

### CROSS-REFERENCE TO RELATED APPLICATION

The present application is a continuation of U.S. patent application Ser. No. 11/551,944, filed Oct. 23, 2006, which claims the benefit of U.S. Provisional Application Ser. No. 60/729,139 filed on Oct. 21, 2006, which is hereby incorporated by reference in its entirety.

### BACKGROUND

The initial treatment for accident victims or emergency room patients often require rapid infusion of intravenous (IV) medication or other treatment-dependent fluids in order to stabilize the patient following a trauma or other serious condition. Once the patient becomes stable, however, the IV infusion rate may be desired to be reduced. It is inefficient and often difficult to replace a rapid IV infusion set with a different IV set having a slower or more normal infusion rate for stable or longer-term use with a particular patient.

### SUMMARY

In one embodiment of the disclosure, an IV infusion apparatus incorporates two drip chambers associated with different drip volume rate nozzles. One nozzle and drip chamber is designed to deliver a rapid infusion rate drip, while the other nozzle and drip chamber is designed to deliver a slower infusion rate drip. A unique switchable valve arrangement allows the infusion rate to be chosen to serve the needs of a patient at any given time.

### DESCRIPTION OF THE DRAWING

FIG. 1 is a perspective view of an intravenous infusion set in accordance with an embodiment of the disclosure.

FIG. 2 is a front elevational view of a valve assembly incorporated as part of the infusion set shown in FIG. 1.

FIG. 3 is a side elevational view of the valve assembly shown in FIG. 2.

FIG. 4 is a top plan view of the portion of the valve assembly shown in FIG. 2.

FIG. 5 is a front elevational cross-sectional view of a valve assembly such as that shown in FIG. 2, taken along line 5-5.

### DETAILED DESCRIPTION

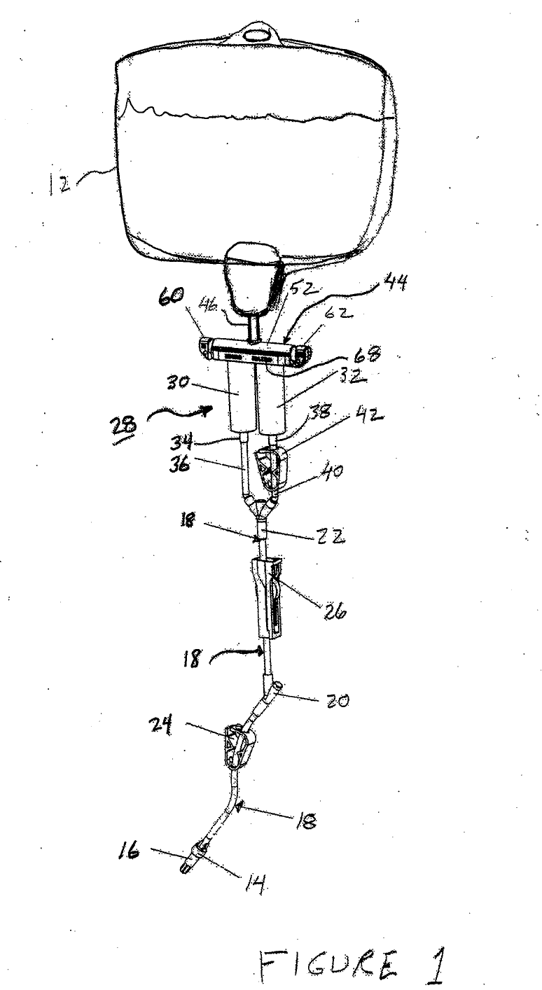

Referring to FIG. 1, there is shown an intravenous (IV) infusion set or apparatus 10, as will be described, which is designed to administer fluids from a fluid container, such as an IV bag 12, to a patient's vascular system through a needle or catheter inserted into a vein for use typically under gravity conditions. Infusion device or apparatus 10 is shown as incorporating a male luer or connector 14 protected by cap 16. Connector 14 is designed to connect to a female luer or connector (not shown) which is associated with or connected to the needle or catheter (also not shown) that would be inserted into a patient's vein. Coupled to connector 14 is tubing 18 which illustratively is shown as including Y-site 20, and Y-connector 22. Y-site 20 provides a location at which additional medications or drugs may be administered by syringe, for example, into tubing 18 for delivery to the patient. A pinch clamp 24 and a roller clamp 26 are also shown as illustratively being located along or coupled to tubing 18.

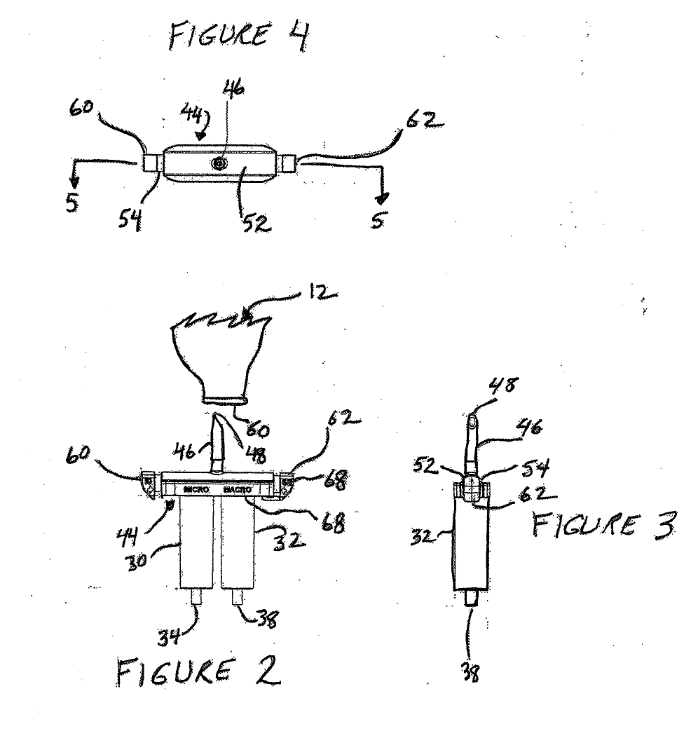

In accordance with an aspect of the disclosure, an infusion rate apparatus 28 comprises drip chambers 30 and 32. The exit 34 of drip chamber 30 is coupled via tubing 36 to one branch of Y-connector 22. The exit 38 of drip chamber 32 is coupled via tubing 40 to the other branch of Y-connector 22. A pinch claim 42 is illustratively shown in place along tubing 40. A valve assembly 44 is positioned in the vicinity of the upper ends of drip chambers 30 and 32. A connector 46 extends upwardly from valve assembly 44 and, as shown more clearly in FIGS. 2 and 5, incorporates a pointed end or spike 48 which is able to pierce the fluid delivery outlet seal 50 of IV bag 12.

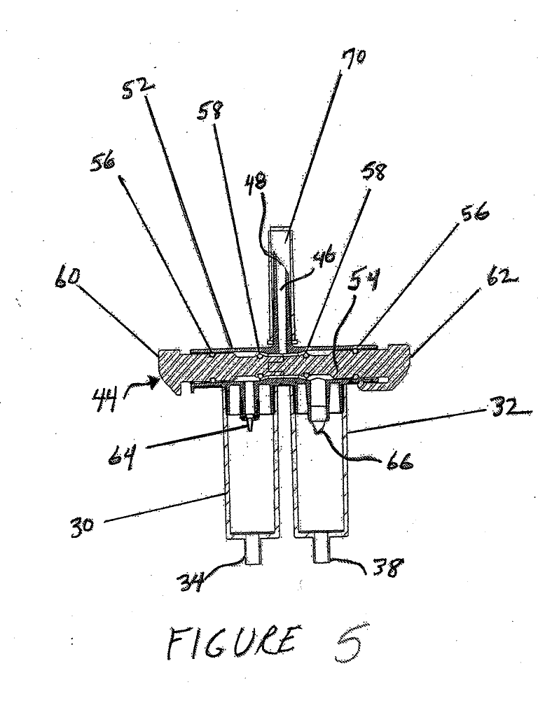

As can be seen in FIGS. 2-5, valve assembly 44 comprises a housing 52 from which connector 46 extends. Housing 44 encloses a selector 54 which slides within housing 52. Selector 54 is illustratively shown as being made of two pieces, but other forms of construction are also possible. O-rings 56 and 58, illustratively constructed of silicone rubber or other suitable material, are shown positioned between selector 54 and housing 52 in order to maintain a fluid-tight seal such that selector 54 operates as a valve piston. O-rings 56 and 58 prevent fluid from entering into the drip chamber that is not being used at the time and they also prevent the fluid from leaking to the outside of the valve assembly 44. The sliding position of selector 54 is chosen to couple connector 46 (which is operatively located within the interior of IV bag 12 as shown in FIG. 1) to either drip chamber 30 or 32. An intermediate position may be permitted that couples connector 46 to both drip chambers as a non-operative “purge” position. In operation, however, selector 54 will either be slidingly moved to contact either end portion 60 or end portion 62 of selector 54 with housing 52, thus stopping the movement of selector 54 in one operating position or the other. In the position in which connector 46 is coupled to drip chamber 30, fluid from IV bag or reservoir 12 flows into drip chamber 30 through a drip volume rate nozzle 64, thereafter flowing from drip chamber 30 via exit 34. In the position in which connector 46 is coupled to drip chamber 32, fluid from IV bag or reservoir 12 flows into drip chamber 32 through a drip volume rate nozzle 66, thereafter flowing from drip chamber 32 via exit 38. Infusion apparatus 10 therefore offers a flow rate selection which allows for the choice of one of two flow volume rates. In the illustrative, non-limiting embodiment shown, flow rates of 10 drops/ml flow volume via drip rate nozzle 66 (designated as macro flow) and 60 drops/ml flow volume via drip rate nozzle 64 (designated as micro flow) are provided by device 10 through the operation of valve assembly 44. Selector slide 54 may be labeled by indicia 68 with the appropriate flow rate, e.g., as in the illustrated embodiment, with a numeral “60” and a numeral “10” along with adjacent, associated arrows indicating the proper direction to push or slide selector 54.

Housing 52 and selector 54 are illustratively shown as being constructed of an ABS material. O-rings 56 and 58, as described above, may be constructed of an FDA-approved silicone material, for example. As illustrative examples only, parts of apparatus 10 may be constructed of the materials as follows, but other suitable materials having the desired operational characteristics may also be effectively used. For example, housing 52 may be constructed of Lustran 348 ABS, O-Rings 56 and 58 may be made of 5568A 010 Silicone, drip chambers 32 and 34 may be constructed of PVC AM88, tubing 18 may be made of PVC Clearflo 6811-02, Y-site 20 may be constructed of ABS Terlux 2802TR, and male luer 14 may be made of Acrylic, such as Perspex CP927G. In the illustrative example of infusion apparatus 10, there are no non-bonded connectors. All bonded connections perform at greater than 15 psi. All connector sites are UV bonded to prevent leakage. The rate that the fluid passes through tubing 18 is controlled by roller clamp 26. The maximum volume rate for apparatus 10 is illustratively 10 drops/ml, while the minimum volume is zero drops/ml (i.e., when fluid flow through apparatus 10 is operatively turned off.

The embodiment of intravenous infusion set or apparatus 10 shown in FIG. 1 is illustratively shown as a disposable, single-use device intended for use under gravity conditions, although other modes of operation and use may be employed as well. Apparatus 10 is intended to be packaged as a sterile item, with sterilization being illustratively performed via exposure to gamma radiation.

The following steps describe one way in which apparatus 10 may be used in connection with the administration of an intravenous solution to a patient. Other steps may be added or used in place of particular described steps as desired in individual applications or situations.

1. Open package (not shown) and remove sterile apparatus 10.

2. Remove end protector 70 from spike 48 and insert spike 48 fully into outlet seal 50 of solution container 12.

3. Verify that the selector 54 is in its center, or “purge,” position.

4. Gently squeeze and release macro and micro drip chambers 30 and 32 until half filled. Do not overfill chambers. Allow solution to clear all air in tubing 18, 36, and 40.

5. Remove the end cap 16 and attach the male luer 14 to the patient's needle or catheter (not shown).

6. Choose the desired flow rate, either the 60 drip/ml micro flow, or the 10 drip/ml macro flow, depending upon patient conditions.

7. Position selector 54 to the desired flow volume position. If 60 drips/ml is desired, push the selector slide in the direction as indicated by its corresponding indicia 68, e.g., the appropriate arrow. If 10 drips/ml is desired, push the selector/slide in the direction as indicated by its corresponding arrow.

8. If 60 drips/ml is chosen and selected, close the pinch clamp 42 located directly below the 10 drip/ml drip chamber 32. This pinch clamp 42 must be reopened if the flow is switched back to the 10 drips/ml flow rate setting.

9. Adjust the fluid flow drip rate by using the roller clamp 26. When a faster drip rate is desired, slowly open the roller clamp 26. When a slower drip rate is desired, slowly close the roller clamp 26.

10. Compatible medication can be injected through Y-site 20, for example.

What is claimed is:

1. An intravenous infusion apparatus comprising:
a connector adapted for coupling to a receptacle containing a quantity of fluid designed for intravenous administration to a patient;
a first drip chamber comprising a first fluid delivery nozzle for delivering said fluid into said first drip chamber at a first rate;
a second drip chamber comprising a second fluid delivery nozzle for delivering said fluid into said second drip chamber at a second rate;
tubing coupled to said first and second drip chambers and adapted for delivering said fluid from said first and second drip chambers to said patient; and
a switch for selectively coupling said connector to said first fluid delivery nozzle or to said second fluid delivery nozzle.

2. The apparatus of claim 1, wherein said switch comprises:
a housing; and
a sliding valve disposed within said housing, said valve having a first delivery position cooperating with said first fluid delivery nozzle, a second delivery position cooperating with said second fluid delivery nozzle, and a purge position cooperating with said first and second fluid delivery nozzles.

3. The apparatus of claim 1, wherein said first fluid delivery nozzle is configured to deliver said fluid at a rate of approximately 60 drips per milliliter.

4. The apparatus of claim 1, wherein said second fluid delivery nozzle is configured to deliver said fluid at a rate of approximately 10 drips per milliliter.
